(19) United States
(12) Patent Application Publication
(10) Pub. No.: US 2014/0276813 A1
(43) Pub. Date: Sep. 18, 2014

(54) SUCTION ELECTROCAUTERY DEVICE HAVING CONTROLLED IRRIGATION AND ROTATING AUGER

(71) Applicant: Larry Kyle Gambrell, Santa Rosa, CA (US)

(72) Inventor: Larry Kyle Gambrell, Santa Rosa, CA (US)

(21) Appl. No.: 13/840,714

(22) Filed: Mar. 15, 2013

(51) Int. Cl. A61B 18/14

(52) U.S. Cl. CPC A61B 18/149
USPC 606/49

(57) ABSTRACT

The present invention is an improved electrocautery device having simplified irrigation controller, a controlled irrigation system and powered rotating auger. Both the rotating auger and the irrigation flow can be turned on and off by operating a controller on the hand-held portion of the device. The auger is part of a flexible rotating drive shaft within malleable suction coagulator conduction tubing. The controller on the handle operates the motor that powers the rotating drive shaft. The motor can also power an irrigation gate to control the flow of fluids through the device's irrigation channel. As an alternative, the irrigation flow can be controlled by mechanical depression of the controller on the handle. The motor can receive power from an AC or DC power source and can be located externally or within the housing of the suction coagulator.

### FIELD OF THE INVENTION

The present invention relates to a surgical device. More specifically, the invention is directed to a suction electrocautery/coagulator device having a one-touch controlled irrigation and rotating auger for tissue resection/removal with or without concurrent coagulation/electroablation.

### BACKGROUND OF THE INVENTION

Suction electrocautery devices, also known as a suction “bovie”, suction electrocautery or suction coagulator, are well known in the art. The suction electrocautery devices typically include an apparatus for suctioning liquids or tissue via a sheathed-electroprotected tube that also performs electrocautery or coagulation at the distal tip. These devices do not actively dissect, resect or otherwise transport the tissue or fluids through the coagulation tube. The standard devices are used primarily for suctioning and coagulating without a primary mechanism for dissection or removal of tissue. However, adenotonsillectomy and other surgical procedures require both the cutting away/resection of tissue, blunt dissection of tissue and ability to concurrently coagulate, electrocauterize or electroablate the tissue. Some electrocautery devices have been modified to perform both cutting and dissection of tissue.

An example of one such suction coagulator is a device having a beveled edge that allows for sharp tip electrocautery, blunt dissection, and suction. Other electrosurgical instruments disclosed in the prior art, not necessarily designed for tonsillectomy procedures, have distinguishing features. For example, a known electrosurgical apparatus includes a rotary, tissue-affecting device in the form of one or more rotating blade(s). The rotating blade of this apparatus is the active energy-delivering electrode.

Other electrosurgical instruments include irrigation, but do not disclose any control of the flow of the liquid therethrough. For example, a known device includes a coagulation mechanism and suction mechanism. The device is configured with an elongate member having an insulative covering attached about a mechanism for coagulating soft tissue. Openings through the covering expose regions of the coagulation-causing elements and are coupled to lumens in the elongate member that are routed to a vacuum source. The device further includes an uncontrolled irrigation system that passively transports fluid along the contacted soft tissue surface.

Another known electrosurgical electrode is configured for use in a tonsillectomy procedure, wherein the electrode incorporates a suction or irrigation conduit. In that device, however, the conduit supplying the suction or liquid is the same conduit. Yet another known cautery instrument with irrigation and suction performs suction and irrigation through the same cannula of the instrument.

The prior art includes a surgical instrument having suction mechanism and an inner rotating cutting member. That instrument is configured with an outer, non-rotating tube in a helical shape for the purpose of bending the instrument. The instrument is also configured with an uncontrolled irrigation line. Similarly, the prior art includes an electrosurgical instrument for performing various oropharyngeal operative procedures. That instrument is configured with a serrated chisel tip for dissecting, an electrocauterizer, a suctioning feature, and an irrigation feature, without control of the irrigation line.

The prior art further includes an electrosurgical instrument having at least two elongate hollow tubes, with one tube being disposed within the other. A cutting tool at a distal end of the inner tube uses heat provided by a radio frequency signal to cut tissue, and the cut tissue is removed under suction through the inner tube. The entire inner tube is energized and rotates. The inner tube is not an auger. A third (irrigation) tube located between the inner and outer elongate tubes of the instrument can feed saline to the cutting area. This electrosurgical instrument is not configured for controlling the fluid delivery of the irrigation line.

Likewise, an electrosurgical probe is known that includes an electrode, suction means, and irrigation means that can be used for oral, otolaryngological, laparoscopic, and dermatologic procedures. The main tube portion of the probe has an electrode at the end and is adapted for suction. The probe can include an irrigation tube, but control of the vacuum and irrigant flow are conventional and not elements of the device.

Another known electrosurgical device is configured with suction, transmission of irrigation fluid, and electrosurgical current, but is configured without a resection mechanism. The device has two different lines to attach to suction and irrigation sources, wherein the lines convene into one line in the device's hand-piece. The electrosurgical current is controlled by a switch on the hand-piece, and the suction and irrigation are controlled by two separate hand-operated control valves that are conduit collapsing mechanisms.

Also known in the prior art is an instrument for removing tissue including a rotatable fluted cutter member housed in a probe adapted to be inserted into a portion of a body from which tissue is to be removed. The tissue is removed via an evacuation conduit. The fluted cutter is attached to a motor within the housing of the instrument. The switching circuit for controlling the motor, however, is remote from the instrument. The instrument can supply irrigation fluid through the probe to the area being operated upon, and can evacuate the material through the probe after being engaged by the cutter. A remote electrical circuit controls the irrigation and suction mechanisms.

Despite the very large number of prior art devices in the field of electrosurgical instruments, none of the devices are believed to include a suction electrocautery instrument having a controlled irrigation system and controlled powered auger for resection, wherein both the irrigation and auger for resection are operated via one controller. A suction electrocautery device with these improvements would allow a surgeon to cauterize tissue, while controlling tissue resection and the delivery of fluid to the surgical site at the same time, with the push of a button. The surgeon could also simultaneously control suction to remove tissue.

Accordingly, there is a need for, and what was heretofore unavailable, is an improved electrocautery device having such features and an enlarged conduction tube for increased tissue removal. The present invention satisfies these and other deficiencies associated with known electrocautery devices.

### SUMMARY OF THE INVENTION

The present invention is directed to a suction electrocautery device having controlled irrigation and a powered rotating auger. Overcoming the limitations of the prior art, the electrocautery device of the present invention may be configured with a simply operated suction apparatus, a controlled irrigation system and a powered auger that allows for more controlled, effective, and less invasive surgery.

The electrocautery device of the present invention allows a surgeon to minimize bleeding during surgical techniques to improve visualization of the surgery and control the patient's blood loss with the electrocautery system. At the same time, irrigation to the site can also improve visualization of the surgery and lower the temperature of the surgical area. In accordance with a method of present invention, a rotating auger may be used to cut tissue so as to decrease operating time and post-operative pain. The electrocautery device of the present invention may further include a fixed post across a cauterizing tip to transect tissue. Thus, the suction electrocautery device of the present invention is an all-in-one multipurpose surgical instrument that can be used for controlled cautery, resection, transection, irrigation, and tissue removal.

Herein, the term “proximal” refers to the portion closest to the user (surgeon), and the term “distal” refers to the portion furthest away from the user (surgeon) and towards the patient anatomy to be affected by the surgery. Also herein, the singular includes the plural and the grammatical articles “or”, “and” may be conjunctive.

The improved suction electrocautery device of the present invention may be configured with a handle having a top portion, a bottom portion, a proximal end, and a distal end. The handle may be further configured with a controller on the top portion of the handle and a suction port at the proximal end of the handle. The suction port is capable of being coupled with an external vacuum source. A finger port is disposed within the top portion of the handle. The handle is configured so that a surgeon can place his or her finger on and off the finger port to control suction through a channel in elongated tubing attached to a distal end portion of the handle and out through a suction port configured in the proximal end portion of the handle.

The electrocautery device is configured with an elongated tubing portion having a suction channel formed therein. The tubing is attached proximate the distal end of the handle and includes an inner conduction portion (for example, formed from a metal) operably connected to an external electrical source. The elongated tubing is configured with a proximal end portion, a distal end portion, and an outer insulating layer extending from the proximal end portion of the tubing proximate the distal end portion of the tubing. The distal end of the tubing is configured with an exposed cautery tip portion, which is formed from a metal or other suitable material. The tubing material may be semi-rigid while being substantially flexible so as to retain a particular shape that aids in the user's approach to the anatomy subjected to treatment.

The electrocautery device of the present invention may control a motor operably connected to a rotating drive shaft that may be positioned within the elongated metal conduction tubing. Both the rotating drive shaft and the elongated tubing may be configured from flexible materials. Further, the electrocautery device may include an irrigation channel configured to be coupled with a fluid source, for example, saline. The irrigation channel may be partially housed within the handle. In accordance with the present invention, the irrigation channel may be configured with an irrigation gate or other suitable apparatus to control flow of fluids through the irrigation channel. The electrocautery device of the present invention may be configured with a controller that simultaneously operates the drive shaft motor and irrigation gate. Thus, a surgeon can operate the controller to simultaneously control tissue resection using the rotating drive shaft while controlling the flow of fluids using the irrigation gate.

In another aspect of the present invention, the rotating drive shaft of the suction electrocautery device may be configured with an auger. The auger may extend substantially the entire length of the rotating drive shaft or may be a positioned on a distal portion of the rotating drive shaft. The auger may be configured with at least one blade capable of tissue resection.

The electrocautery device of the present invention may be further configured for transecting tissue using a fixed post across an exposed cautery tip positioned proximate the distal portion of the elongated tubing. The fixed post may be attached to or configured from the conductive material within the elongated tubing.

In yet another aspect of the present invention, the distal end portion elongated tubing of the electrocautery device may include at least one reinforcement section for support, for example, when the electrocautery device is used in a bent position. Alternatively, the rotating drive shaft may be configured with at least one member for supporting and positioning the rotating drive shaft within the elongated tubing.

The motor of the suction electrocautery device may be housed within or outside of the handle portion. The motor may be powered by an alternating current (AC) or direct current (DC) power source, for example, a battery power source. In an alternative embodiment of the present invention, the electrocautery device may be configured with an air-driven motor.

The controller of the suction electrocautery device may be configured to simultaneously electrically operate the rotating drive shaft and mechanically operate the irrigation gate. Alternatively, the controller may electrically operate both the rotating drive shaft and the irrigation gate by using the motor. The motor may be configured to power a gearbox or solenoid mechanism to open and close the irrigation gate.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a side view in partial cross-section of a suction electrocautery device according to the present invention.

FIG. 2 is a side orthogonal view of a suction electrocautery device according to the present invention.

FIG. 3 is a top orthogonal view of a suction electrocautery device according to the present invention.

FIG. 4 is a bottom orthogonal view of a suction electrocautery device according to the present invention.

FIG. 5 is an orthogonal view of a cross-section of an embodiment of the distal portion of a suction electrocautery device according to the present invention.

FIG. 6 is a side view in cross-section of a first embodiment of an elongated tubing portion of a suction electrocautery device according to the present invention.

FIG. 7 is a side view in cross-section of a second embodiment of an elongated tubing portion of a suction electrocautery device according to the present invention.

FIGS. 8A and 8B are views in cross-section of an electrically operated irrigation gate configured with a spring actuator according to one aspect of the present invention.

FIGS. 9A and 9B are views in cross-section of an electrically operated irrigation gate configured with a gear actuator according to one aspect of the present invention.

FIG. 10A is a end view in cross-section of a mechanically operated irrigation gate according to one aspect of the present invention.

FIGS. 10B and 10C are top views in cross-section of the mechanically operated irrigation gate shown in FIG. 10A.

FIG. 11 is a schematic representation of an alternative embodiment of an irrigation gate for use with the suction electrocautery system of the present invention.

### DETAILED DESCRIPTION OF THE INVENTION

As shown in the drawings for purposes of illustration, the present invention is directed to a new and improved suction electrocautery device for surgical procedures. The suction electrocautery device of the present invention may be configured with a motorized rotating auger and a gate-controlled irrigation channel. An operator-controlled button may be included to simultaneously control both the operation of the rotating auger and the operation of the irrigation channel gate.

Referring to FIG. 1, the suction electrocautery device 10 of the present invention includes a handle portion 20 having a top portion 22, a bottom portion 24, a proximal end 26, and a distal end 28. The handle 20 may be configured with a suction port 30 positioned at the distal end of the handle 20. The suction port 30 may be configured to be coupled with a vacuum source external to the electrocautery device 10. The suction mechanism of the electrocautery device 10 can thereby remove tissue from the surgical area by connecting the suction port 30 to a vacuum source using a suction tube our other suitable apparatus.

The handle 20 may also include a finger port 32 to control suction of gases, liquids or other materials through the electrocautery device 10. As shown in FIG. 3, the finger port 32 may be configured as an aperture disposed in the top portion 22 of the handle 20. The finger port 32 of the present invention may be configured to function similar to or the same as those of suction bovies heretofore described and currently used by surgeons. The electrocautery device handle 20 may be configured so that a surgeon can cover the finger port 32 with his or her thumb or finger. When the finger port 32 is covered, then the vacuum pressure in the suction chamber 34 increases so as to draw air through the distal tip 46 of the elongated tubing 40, through the suction channel 42 and out through the suction port 30 configured within the handle 20. Thus, gas, liquids and other solid and semi-solid materials are removed from the surgical area. During suction, the surgeon's finger or thumb may be protected from electric shock by methods known by those of ordinary skill in the art of making medical devices. When a surgeon removes his or her finger or thumb from the finger port 32, air is drawn through the finger port 32, reducing the pressure and decreasing the airflow drawn from the distal tip portion 46 of the elongated tubing 40. Accordingly, the suction mechanism of the electrocautery device 10 can be precisely controlled by the surgeon with a single touch of his or her hand.

As illustrated in FIGS. 1-4, the suction electrocautery device 10 of the present invention includes an elongated tubing 40 portion connected to the distal end portion 28 of the handle 20. The elongated tubing 40 includes proximal end portion 44 and a distal end portion 46. The elongated tubing 40 includes and inner portion 45 formed form a conductive material, such as, but not limited to, medical grade metals known to one of ordinary skill in the art. The conductive inner portion 45 of the elongated tubing 40 may be surrounded by an outer insulating layer 48. The insulating layer 48 extends from the proximal end portion 44 of the tubing 40 to proximate the distal end portion 46 of the tubing 40.

A contemplated with the electrocautery device 10 of the present invention, the proximal end portion 44 of the elongated tubing 40 is operably connected to an electricity source connector 49 configured in the proximal portion 26 of the handle 20 so as to provide electric current to the inner conductive portion 45 of the elongated tubing 40. The electricity source connector 49 may be configured to connect to an AC or DC power source, such as those used with known electrocautery devices.

As shown in FIG. 5, the distal end portion 46 of the elongated tubing 40 may be configured with an exposed metal cautery tip 50 that is not insulated by the outer layer 48. The cautery tip 50 can include a fixed post 52 across an opening at the distal portion 46 of the elongated tubing 40. The fixed post 52 can thereby used, as a non-limiting example, for transecting lymphoid tissue. The fixed post 52 is configured in a fixed position relative to the cross-section of the elongated tubing 40 and distal end 74 the auger 72, for example, while the auger 72 rotates and resects tissue (see FIGS. 6 and 7).

The elongated tubing 40 of the electrocautery device 10, including the exposed metal cautery tip 50, is configured with a suction channel (lumen) 42, which may be formed by configuring the conductive portion 45 as a hollow tubular member. The suction channel 42 is fluidly coupled with a suction port 30 configured within the handle 20 so as to connect with a vacuum source as used with known suction electrocautery devices and within the ordinary skill in the art of manufacture medical devices. As shown in FIG. 5, the fixed post 52 creates a bifurcated opening 47 in the channel 42 at the distal portion 46 of the elongated tubing 40.

The conductive material used in the elongated tubing 40 is preferably a long-lasting, light flexible material that facilitates efficient conduction of electricity from the handle portion 20 through the conductive portion 45 to the cautery tip 50. The conductive portion 45 of the elongated tubing 40 can be formed of any suitable material, such as medical grade metals. The insulating layer 48 can be formed a coating made of various known electrical isolation materials, such as PVC, rubber or any other suitable isolation material to avoid electrical shock or burns.

As is known regarding suction coagulators currently used by surgeons and described in the prior art, the diameter of the elongated tubing 40 portion of the electrocautery device 10 of the present invention can be enlarged for increased tissue removal. Similarly, the diameter of the elongated tubing 40 may enlarged to retain a rotating drive shaft 70 therein (see FIGS. 6 and 7). The elongated tubing 40 can be made in various lengths and diameters as required by the use of the surgeon and the target anatomy of the patient. Preferably, the elongated conduction tubing 40 is approximately fifteen centimeters long and four millimeters wide.

In addition to being attached with the distal end 28 of the handle 20, the conductive portion 45 in the proximal end 44 of the tubing 40 is operably connected with an electrical connector 49 disposed within the handle 20. In accordance with the present invention, the electrical connector 49 of the electrocautery device 10 may be configured for a conventional method of powering the cautery tip 50. As shown in FIG. 1, the electrical source 49 can be an electrical cord that connects the conductive portion 45 within the elongated tubing 40 with an electrical power source. The electrical power source can thereby activate the exposed metal cautery tip 50 via the electrical cord 49, and the cautery tip 50 can thereby be used for dissection. At the same time, suction can be applied through a suction port 30 to remove liquid or other materials at the dissection or cauterizing area through the channel 42 in the elongated tubing 40. The electrical source 49 is electrically isolated from the suction port 30 in a conventional manner as is used in known suction electrocautery devices and to one of ordinary skill in the art.

As shown in FIGS. 1, 5, 6, and 7, a rotating drive shaft 70 is mounted within the elongated tubing 40 portion of the electrocautery device 10 and is operably coupled with a motor 60 disposed within the handle portion 20. Both the rotating drive shaft 70 and the elongated tubing 40 are made of suitable malleable, yet sturdy, materials. The malleable materials allow the rotating drive shaft 70 and elongated tubing 40 to be positioned and stably held at different angles. As illustrated in FIG. 6, one or more support members 120 can be attached to the flexible rotating drive shaft 70 at certain points within the channel 42 of the elongated tubing 40 so as to fit within the interior circumference of the conductive material 45. The support members 120 reinforce the stability of the rotating drive shaft 70 when it and the elongated tubing 40 portion are bent or at differently angled positions. In the alternative, as illustrated in FIG. 1, the elongated tubing portion 40 may be configured with at least one reinforced section 110 at certain point(s) along the inner channel 42 so as to support the tubing 40 when it is in a bent position at the reinforced sections 110. During normal use of the electrocautery device 10, the rotating drive shaft 70 and elongated tubing portion 40 will be in a bent position or in any desired position required to access the surgical site.

In a contemplated embodiment of the present invention of a suction electrocautery device 10 of the present invention, the rotating drive shaft 70 can also be confidential with an auger 72. As shown in FIG. 7, the entire length of the rotating drive shaft 70 can be in the form of an auger 72. Alternatively, as shown in FIGS. 1 and 6, only the distal end portion 76 of the rotating drive shaft 70 includes an auger 72. In either the auger tip embodiment (FIG. 6) or the full-length auger embodiment (FIG. 7), the distal end 74 of the auger 72 can be configured as least one sharp blade that for tissue resection. The auger 72 can also guide the resected tissue through the channel 42 of the elongated tubing 40 for improved tissue removal. The auger 72 therefore assists both the cauterized metal tip 50 for cutting and the suction mechanism 30, 32, 34 for tissue removal.

Referring to FIG. 1, the proximal end of the rotating drive shaft 70 is operably connected to a motor 60. More specifically, the axis of the drive shaft 70 can be aligned with the axis of a motor shaft via a conventional gear box 130. The motor 60 is electrically isolated from the portions of the handle 20 of the electrocautery device 10 that contact the surgeon. Alternatively, the motor 60 may be positioned may be positioned outside the device handle 20. An aspect of housing the motor 60 inside the handle 20 is that the motor axis and axis of the drive shaft 70 are aligned. An advantage of the motor being outside of the handle is that the hand-held portion may be configured be thinner so as to provide better electrical isolation of the motor. The motor 60 may be operated using alternating current (AC) source or direct current (DC) source. Alternatively, the electrocautery device 10 of the present invention may be configured with and air-driven motor 60 for added safety. As a non-limiting example, as shown in FIG. 1, the motor 60 may be powered from the same source connector 49 for electric current as that of the conductive portions 45, 50 of the electrocautery device 10. In the alternative, the motor 60 may be powered through a DC adapter supply from an external power source (not shown) for the electrocautery device 10. Consistent with the present invention, the motor 60 may be powered by rechargeable or disposable batteries.

As illustrated in FIG. 3, the handle 20 of the electrocautery device 10 includes a controller 100, such as a switch or button. The controller 100 can simultaneously mechanically operate an irrigation gate 90 (see FIGS. 8A-10C) while electrically operating the rotating drive shaft 70 via the motor 60. Alternatively, the controller 100 can simultaneously electrically operate both the irrigation gate 90 and the rotating drive shaft 70 via the motor 60. The controller 100 may be electrically connected with the motor 60 to provide or remove electrical power to the motor 60. When the motor 60 receives electricity by, for example, depressing the controller 100, both the irrigation gate 90 (via either mechanical or electrical operation) and the rotating drive shaft 70 (via electrical operation) are provided power to operate.

To operate the rotating drive shaft 70, the motor 60 is connected with a conventional gear box 130. As shown in FIG. 1, the gear box 130 can have a motor shaft that is coupled with the proximal end of the rotating drive shaft 70. Thus, when the surgeon operates the controller 100, the controller 100 will power on the motor 60, which will rotate the motor shaft of the gear box 130. The rotating motor shaft of the gear box 130 thereby rotates the rotating drive shaft 70 connected therewith. Methods of operating the irrigation gate 90 by the controller 100 are described below.

As shown in FIGS. 1-4, the electrocautery device 10 of the present invention may include an irrigation channel 80 configured along the outside length of the insulated portion 48 of the elongated tubing 40. The proximal portion of the irrigation channel 80 may be partially housed within the handle portion 20. In particular, the proximal portion of the irrigation channel 80 is partly housed within the handle 20 and is secured or otherwise formed along the outside of the length of the insulated portion 48 of the elongated tubing 40. The proximal end portions and distal portions of the irrigation channel 80 are configured to deliver fluid to the distal end 46 of the elongated tubing 40. The irrigation channel 80 can use a standard I.V. connection to a fluid source and can utilize a standard I.V. flow reducer to control the gravity flow of liquid through the irrigation channel 80. Accordingly, the irrigation channel 80 is isolated from the electrical components of the electrocautery device 10. The irrigation channel 80 can have any suitable diameter in relation to the diameter of the elongated tubing 40, wherein two millimeters is a non-limiting example. The irrigation channel 80 can be grooved.

The flow of the irrigation channel 80 can be controlled in several manners via an irrigation gate 90. The irrigation gate 90 can be controlled electrically by the motor 60 or by mechanical operation of the controller 100. Variations of the flow control of the irrigation channel 80 via the irrigation gate 90 are shown by magnified illustrations in FIGS. 8A-10C. FIGS. 8A, 8B, and 9 show electrically controlled irrigation and FIGS. 10A-10C show mechanically controlled irrigation.

One embodiment of the electrically controlled irrigation gate 90 uses an electromagnetic solenoid mechanism. For example, as shown in FIG. 8A, a pin 140, which is the armature of the solenoid 150, blocks flow of liquid through the irrigation channel 80 when it is in a resting position because it is held in place in the blocking position by a spring 160. As shown in FIG. 8B, the solenoid 150 is electrically activated when the motor 60 is powered on by the controller 100. The solenoid 150 converts electrical energy into mechanical energy to move the pin 140 to open or close the irrigation gate 90. When the operator turns on the motor 60 via the controller 100, the pin 140 is drawn in a direction away from the irrigation channel 80 via a magnetic force from the solenoid 150, compressing the spring 160. Thus, liquid can flow freely past the irrigation gate 90 because the pin 140 is not blocking the irrigation channel 80. Once the motor 60 is turned off by the controller 100, the pin 140 and spring 160 return to the resting position, blocking flow of liquid through the irrigation channel 80.

Another embodiment of the electrically controlled irrigation gate 90 uses a gear box 130 powered by the motor 60. As shown in FIG. 9, the motor 60 is coupled with a pin 140 via a slip gear 180 of the gear box 130. When the motor 60 is off, the pin 140 is blocking the flow of the irrigation channel 80 via a spring mechanism 160. When the motor 60 is powered on by depressing the controller 100, the slip gear 180 of the gear box 130 coupling the pin 140 to the motor 60 will move the pin 140 away from the irrigation channel 80, compress the spring 160, and thus open the flow through the irrigation channel 80.

An embodiment of the non-electric, mechanically controlled irrigation gate 90 is shown in FIGS. 10A-10C. The mechanical gate is activated by mechanical depression of the controller 100. In this embodiment, a pin 140 is held in place with a spring 160 to block flow of the irrigation channel 80 when the controller 100 is in an initial position. The resting position is shown in FIG. 10B. The pin 140 is mechanically coupled with the controller 100, e.g., through a gear system 190. When the controller 100 is depressed, the gear system 190 rotates to draw the pin 140 away from the irrigation channel 80 so the channel is no longer blocked and fluid can flow freely through the irrigation channel 80. The activated position of the irrigation gate 90 and open irrigation channel 80 are shown in FIGS. 10A (back end view) and 10C (top view).

An embodiment of the non-electric, mechanically controlled irrigation gate 200 is shown in FIG. 11. The gate includes a housing 210 and compressed spring 220 around the pin. A flexible band 230 connects the pin to the fluid mechanism.

While particular forms of the invention have been illustrated and described with regard to certain embodiments of suction electrocautery devices, it will also be apparent to those skilled in the art that various modifications can be made without departing from the scope of the invention. While certain aspects of the invention have been illustrated and described herein in terms of its use with specific content types, it will be apparent to those skilled in the art that the system can be used with many types of procedures not specifically discussed herein. Further, particular embodiments of suction electrocautery devices and systems have been described herein and are provided as examples only. Other modifications and improvements may be made without departing from the scope of the invention.

I claim:

1. A suction electrocautery device, comprising:
a handle having a top portion, a bottom portion, a proximal end, and a distal end, and wherein said handle further comprises a controller on said top portion of said handle and a suction port at said proximal end of said handle, wherein said suction port capable of being coupled with a vacuum source;
a finger port disposed within said top portion of said handle;
an elongated metal conduction tubing having a suction channel formed therein,
wherein said tubing is operably connected with an electrical source and wherein said tubing is attached with said distal end of said handle,
wherein said tubing further comprises a proximal end and a distal end, and an outer insulating layer extending from said proximal end of said tubing to said distal end of said tubing, and wherein said distal end of said tubing further comprises an exposed metal cautery tip portion;
a motor;
a rotating drive shaft operably connected with said motor and positioned within said elongated metal conduction tubing;
an irrigation channel capable of being coupled with a fluid source, wherein said irrigation channel further comprises an irrigation gate to control flow of fluids therethrough,
wherein said controller of said handle operates said motor and said irrigation gate, and
wherein said controller is configured to simultaneously control tissue resection by said rotating drive shaft and control said flow of fluids by said irrigation gate, and whereby a said finger port is configured to control suction through said suction channel and out of said suction port of said device.

2. The suction electrocautery device of claim 1, wherein said rotating drive shaft further comprises an auger.

3. The suction electrocautery device of claim 2, wherein said auger includes substantially the entire length of said rotating drive shaft.

4. The suction electrocautery device of claim 2, wherein said auger comprises a portion of a distal end of said rotating drive shaft.

5. The suction electrocautery device of claim 2, wherein said auger further comprises at least one blade capable of tissue resection.

6. The suction electrocautery device of claim 1, wherein both said rotating driveshaft and said elongated metal conduction tubing are flexible.

7. The suction electrocautery device of claim 1, wherein said device further comprises a fixed post across said exposed metal cautery tip portion at said distal end of said elongated metal conduction tubing.

8. The suction electrocautery device of claim 1, wherein said elongated metal conduction tubing further includes at least one reinforcement section to support said elongated metal conduction tubing.

9. The suction electrocautery device of claim 1, wherein said rotating drive shaft further comprises at least one support member to support said rotating drive shaft.

10. The suction electrocautery device of claim 1, wherein said motor is housed within said handle.

11. The suction electrocautery device of claim 1, wherein said motor is housed outside of said handle.

12. The suction electrocautery device of claim 1, wherein said motor is operably connected with said electrical source and thereby powered by said electrical source.

13. The suction electrocautery device of claim 1, wherein said motor is operably connected with a batter power source.

14. The suction electrocautery device of claim 1, wherein said irrigation channel is at least partially housed within said handle.

15. The suction electrocautery device of claim 1, wherein said controller mechanically operates said irrigation gate.

16. The suction electrocautery device of claim 1, wherein said controller electrically operates said irrigation gate via said motor.

17. The suction electrocautery device of claim 16, wherein said electrical operation is selected from the group consisting of a solenoid mechanism and a gearbox mechanism.

18. A process for performing suction electrocautery, comprising:
providing an electrocautery device including a handle portion attached to an elongated tubing portion and attached to an irrigation channel;
connecting an electrical source to a conductive portion configured within the elongated tubing;
connecting a fluid source connected a gate configured to control fluid flow to the irrigation channel;
coupling a vacuum source to a suction port configured within the handle and connected to a suction channel formed in the elongated tubing;
using a controller configured in the handle to provide power from the electrical source to the conductive portion of the elongated tubing;
using the controller configured in the handle to operate the gate so as to move fluid from the fluid source through the irrigation channel to a distal end of the elongated tubing;
using the controller configured in the handle to provide electric current to a distal tip of the conductive portion of the elongated tubing; and
using a finger port disposed within the handle to control the suction in the channel formed in the elongated tubing.

19. A process for performing suction electrocautery of claim 18, further comprising:
providing a motor and a rotating drive shaft operably connected with said motor; and
using the controller configured in the handle to operates the motor to rotate the drive shaft.

20. A process for performing suction electrocautery of claim 19, wherein rotating the drive shaft further rotates an auger attached to a distal portion of the drive shaft.
